(19) United States
(12) Patent Application Publication
(10) Pub. No.: US 2020/0312443 A1
(43) Pub. Date: Oct. 1, 2020

(54) METHOD AND SYSTEM FOR IDENTIFYING VARIETY IN PATHWAYS AND PERFORMANCE WITHIN AN EPISODE OF CARE

(71) Applicant: KONINKLIJKE PHILIPS N.V., Eindhoven (NL)

(72) Inventors: Daniele DE MASSARI, Eindhoven (NL); Steffen Clarence PAUWS, Eindhoven (NL); Christoph Tobias WIRTH, Velmar (DE); Dieter Maria Alfons VAN DE CRAEN, Balen (BE)

(21) Appl. No.: 16/829,432

(22) Filed: Mar. 25, 2020

Related U.S. Application Data
(60) Provisional application No. 62/823,686, filed on Mar. 26, 2019.

(51) Int. Cl. G16H 40/20; G16H 10/60; G16H 50/70; G06Q 30/04; G16H 70/20

(52) U.S. Cl. CPC G16H 40/20; G16H 10/60; G06N 20/00; G06Q 30/04; G16H 70/20; G16H 50/70

(57) ABSTRACT

Example embodiments include a method for managing healthcare services including receiving information selecting an episode of care, identifying a plurality of care pathways for the episode of care, generating a plurality of clusters corresponding to the plurality of care pathways, respectively, and performing a clustering analysis to determine a first care pathway of the plurality of care pathways, and outputting information indicative of the first care pathway. Each of the care pathways corresponds to services from a combination of different healthcare personnel with associated costs, and the first care pathway corresponds to one of the plurality of clusters satisfying a selected outcome measure for a bundled payment reimbursement contract.

### CROSS-REFERENCE TO PRIOR APPLICATIONS

This application claims the benefit of U.S. Provisional Application No. 62/823,686, filed on 26 Mar. 2019. This application is hereby incorporated by reference herein.

### TECHNICAL FIELD

This disclosure relates generally to processing information, and more specifically, but not exclusively, to managing the allocation and cost of providing healthcare resources.

### BACKGROUND

Healthcare organizations are slowly transitioning from fee-for-service to value-based reimbursement schemes. This transition implies that such organizations are considered accountable for all the healthcare services rendered to patients within a predefined unit of measure. One example involves capitated models that require organizations to be accountable for the health of a selected group of patients within a contract period (e.g. 1 or 3 years), independent of the disease(s) affecting the patient health.

Another example is represented by bundled payments, where the boundaries refer to a specific episode or condition. Consequently, accountability is restricted to a specific need of the patient and not to his general health status, as in the previous example. Bundled payment reimbursement schemas revolve around the concept of episodes of care.
Episodes of care may be categorized into two classes: acute and chronic. An acute episode of care usually involves a surgical procedure (e.g. coronary artery bypass surgery bypass operation, percutaneous coronary intervention, etc.) that is usually bounded to a time period that triggers based on when the episode occurred. The time period may span from 10 days before the surgery (to include any necessary screening or diagnostic procedures prior to the operation) to 90 days after the surgery to capture all post-surgery services (e.g., rehabilitation sessions, medication, etc.). Chronic episodes of care focus on chronic conditions; hence, they have usually a start date (e.g., 30 days before the first diagnosis) but no end date.

Different algorithms have been designed to extract episodes of care from clinical and administrative data. These algorithms differ in the way they identify the starting trigger event, select relevant services for the episode under investigation, identify concurrent medical events that are considered complications caused by a poor management of the selected episode (e.g., potentially avoidable complications PACs)), and attribute the cost of the episode to a provider or distribute the cost among multiple providers.

In the interests of providing quality care for the patient and promoting financial sustainability for the healthcare provider organization, it would be beneficial to have a system and method which is able to provide reliable information indicating the variety of care pathways and performance that exist for any given episode.

### SUMMARY

A brief summary of various example embodiments is presented below. Some simplifications and omissions may be made in the following summary, which is intended to highlight and introduce some aspects of the various example embodiments, but not to limit the scope of the invention. Detailed descriptions of example embodiments adequate to allow those of ordinary skill in the art to make and use the inventive concepts will follow in later sections.

In accordance with one or more embodiments, a method for managing healthcare services includes receiving information selecting an episode of care; identifying a plurality of care pathways for the episode of care, each of the care pathways corresponding to services from a combination of different healthcare personnel with associated costs; generating a plurality of clusters corresponding to the plurality of care pathways, respectively; performing a clustering analysis to determine a first care pathway of the plurality of care pathways, the first care pathway corresponding to one of the plurality of clusters satisfying a selected outcome measure for a bundled payment reimbursement contract; and outputting information indicative of the first care pathway.

Performing the clustering analysis may include performing a clustering analysis to determine the first care pathway. Performing the clustering analysis may include generating a plurality of trajectories for respective ones of the plurality of clusters, wherein each trajectory corresponds to different combination of healthcare services or services from a different combination of healthcare personnel. The plurality of clusters may be generated based on medical information derived from a plurality of healthcare resources. The clustering analysis may include performing a cross-tabulation, stratification, or regression algorithm on the plurality of clusters. The outcome measure may include at least one of volume, quality of care, cost, and variation in costs. The outcome measure may include cost of healthcare services over a period of time under the bundled payment reimbursement contract, and the first care pathway may correspond to a lowest one of the total cost of the healthcare services. Outputting information indicative of the first care pathway may include ranking the plurality of care pathways for the episode of care.

In accordance with one or more embodiments, a system for managing healthcare services includes an interface and a processor configured to identify a plurality of care pathways for an episode of care, generate a plurality of clusters corresponding to the plurality of care pathways, respectively, perform a clustering analysis to determine a first care pathway of the plurality of care pathways, and output information through the interface indicative of the first care pathway, wherein each of the care pathways corresponds to services from a combination of different healthcare personnel with associated costs and wherein the first care pathway corresponds to one of the plurality of clusters satisfying a selected outcome measure for a bundled payment reimbursement contract.

The processor may perform a clustering analysis to determine the first care pathway. The clustering analysis may include generating a plurality of trajectories for respective ones of the plurality of clusters, wherein each trajectory corresponds to different combination of healthcare services or services from a different combination of healthcare personnel. The clustering analysis may include a cross-tabulation, stratification, or regression algorithm on the plurality of clusters. The outcome measure may include cost of healthcare services over a period of time under the bundled payment reimbursement contract, and the first care pathway corresponds to a lowest one of the total cost of the healthcare services.

In accordance with one or more embodiments, a non-transitory machine-readable medium including instructions to cause a processor to identify a plurality of care pathways for an episode of care; generate a plurality of clusters corresponding to the plurality of care pathways, respectively; perform a clustering analysis to determine a first care pathway of the plurality of care pathways; and output information indicative of the first care pathway, wherein each of the care pathways corresponds to services from a combination of different healthcare personnel with associated costs and wherein the first care pathway corresponds to one of the plurality of clusters satisfying a selected outcome measure for a bundled payment reimbursement contract.

The processor may perform a clustering analysis to determine the first care pathway. Performing the clustering analysis may include generating a plurality of trajectories for respective ones of the plurality of clusters, wherein each trajectory corresponds to different combination of healthcare services or services from a different combination of healthcare personnel. The outcome measure may include cost of healthcare services over a period of time under the bundled payment reimbursement contract, and the first care pathway may correspond to a lowest one of the total cost of the healthcare services.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate example embodiments of concepts found in the claims and explain various principles and advantages of those embodiments.

These and other more detailed and specific features are more fully disclosed in the following specification, reference being had to the accompanying drawings, in which:

FIG. 1A illustrates an embodiment of a system for managing healthcare services, and FIG. 1B illustrates an example of process flow for the system of FIG. 1;

FIG. 2 illustrates an embodiment of an episode care pathway analyzer module;

FIG. 3 illustrates an embodiment of a longitudinal clustering algorithm;

FIG. 4 illustrates example results obtained by the longitudinal clustering algorithm;

FIG. 5 illustrates the example results of FIG. 4 ordered and output using a box plot;

FIG. 6 illustrates an embodiment of a cross-sectional clustering algorithm; and

FIG. 7 illustrates an embodiment of a method for managing healthcare services.

### DETAILED DESCRIPTION

It should be understood that the figures are merely schematic and are not drawn to scale. It should also be understood that the same reference numerals are used throughout the figures to indicate the same or similar parts.

The descriptions and drawings illustrate the principles of various example embodiments. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various example embodiments described herein are not necessarily mutually exclusive, as some example embodiments can be combined with one or more other example embodiments to form new example embodiments. Descriptors such as “first,” “second,” “third,” etc., are not meant to limit the order of elements discussed, are used to distinguish one element from the next, and are generally interchangeable. Values such as maximum or minimum may be predetermined and set to different values based on the application. In accordance with one or more embodiments, an “episode of care” may include the collection of all healthcare services rendered to a specific patient to treat a specific condition.

In the United States, bundled payments arrangements are offered by public and private payers for various episodes. These payment arrangements are complex and often subject to various guidelines, programs, and government regulations. Consequently, healthcare organizations (HCOs) are often unable to provide or authorize proper care for patients in a cost-effective manner. In order to improve this situation, it would be beneficial for HCOs to not only understand the whole concept of an episode of care, but also to have at their disposal methods to track operations and determine proper care pathways for any given episode of care.

Example embodiments describe a system and method for partitioning and ranking a plurality of care pathways within a given episode of care. The partitioning and ranking may be performed based on one or more predetermined attributes, including but not limited to patient volume, quality of care, cost of care, variation in spending, and/or outcome. By ranking care pathways in this manner, the care pathway that best satisfies a selected outcome for a given episode may be determined. In one embodiment, the best or optimal care pathway may be identified in order to increase the profit margin of the healthcare provider organization in relation to bundled payment reimbursement contracts.

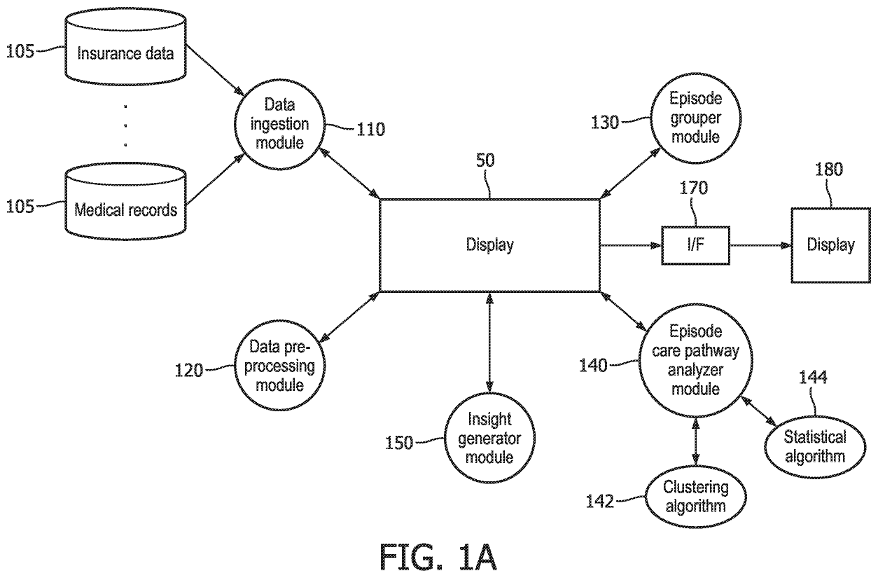

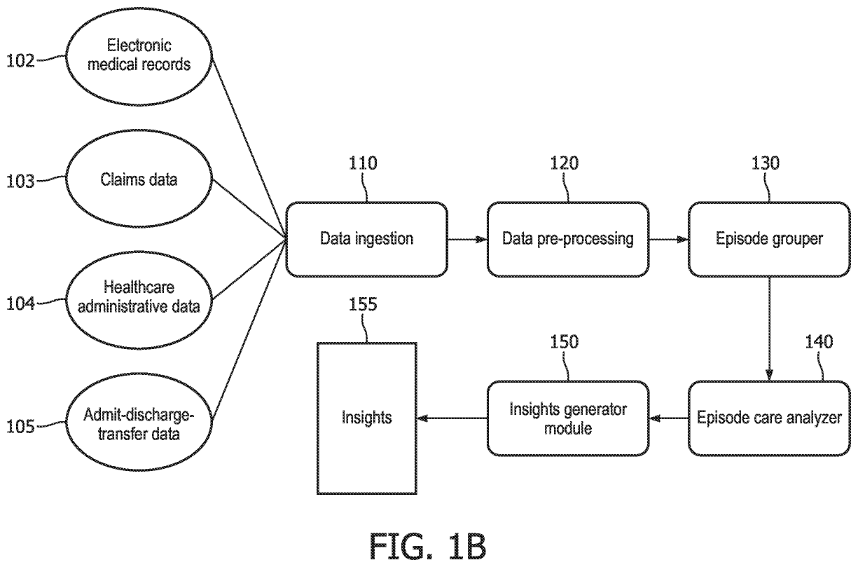

FIG. 1A illustrate an embodiment of a system for managing healthcare services which includes a processor 50 coupled to a data ingestion module 110, a data pre-processing module 120, an episode-grouper module 130, an episode care pathway analyzer module 140, and an insight generator module 150. The modules may communicate with one another through the processor 50 and/or directly with one another. Each module may be implemented in logic that includes hardware, software, or a combination of hardware and software. In a software embodiment, processor 50 executes instructions to perform operations of the modules. FIG. 1B illustrates an example of the arrangement of the modules relative to the processing that may take place by the system of FIG. 1A in accordance with one embodiment.

The data ingestion module 110 connects the processor 50 to different data sources 105. These data sources may store various types of information, including but not limited to electronic medical records (EMRs) 102, insurance claims data 103, healthcare administrative information 104, and admit-discharge-transfer (ADT) 105. The data sources 105 may all reside within the same database or may be distributed throughout different databases connected to the processor through the data ingestion module 110. In one embodiment, some or all of the information from the data sources may be received from a cloud-based network. In these or another embodiments, the data sources may be stored in a blockchain or some other decentralized database managed within a distributed network. The connection to the data ingestion module 110 may be pushed from the data sources and/or may be accessed by the data ingestion module 110 according to a predetermined time schedule or whenever requested. Once received, the data may be streamed to the data pre-processing module 120.

The data pre-processing module 120 may merge, normalize, and pre-process the data from the data ingestion module 110. This may include generating a final claim identification, performing data reconciliation, and/or other operations as described in greater detail below.

The episode-grouper module 130 may extract a starting trigger event, select relevant services, and identify complications for the episode under investigation. Such an episode-grouper module 130 may correspond to a commercially available grouper application which has been modified to group claims and other information relating to episodes. The National Quality Forum (NQF) provides a list of episode groupers that may be used to implement the episode-grouper module.

The episodic care pathway analyzer module 140 analyzes different care pathways for a selected episode of care, groups the care pathways (e.g., cross-sectionally or longitudinally), and identifies a group (e.g., a trajectory group) that satisfies a selected attribute outcome (e.g., outcome, cost, or quality). In one embodiment, the episode care pathway analyzer module 140 may identify the best or optimal group that satisfies a selected attribute outcome. The episode care pathway analyzer module 140 may also perform a cross-tabulation, stratification, and/or regression analysis on one or more (external) variables, including but not limited to beneficiary characteristics.

The insight generator module 150 may report insights 155 derived by the analysis performed by the episodic care pathway analyser module 140. A more detailed description of the modules and their respective operations is provided below.

### Data Ingestion Module

The concept of an episode of care includes identifying all the services offered to a patient which are deemed connected to the episode under investigation, both as typical and avoidable complications. Different sources of data/information may be consulted to collect as complete a picture as possible of all services received by a patient. In one embodiment, one or more of the data sources provide information from Centers for Medicare & Medicaid Services (CMS) source. Examples of the data from the data sources include:
claims data (CMS Claims Line Feed—CCLF) or commercial payer claims.
HASSIGN and QASSIGN files containing preliminary assigned beneficiary list(s) indicating persons who are qualified, newly and previously assigned beneficiaries to an HCO (e.g., on a monthly and yearly basis), persons who are disabled or ESRD (heavy-cost) beneficiaries. This data may be provided with CMS-HCC risk scores from prior years, and data on primary care utilization including those providers with most primary care services provided.
AASR/QASR files including Assignment Summary Reports providing aggregate HCC data, compared with a typical MSSP ACO (HCO) or geographical data of beneficiaries.
QEXPU files being Aggregate Expenditure and Utilization Report providing expenditures and utilization of preliminarily assigned population in previous quarters.
A physician roster file for enumerating all affiliation organizations and physicians within an HCO claims network, both in-network or as out-of-network;
EMR data containing all clinical data of beneficiaries who are qualified, newly or previously attributed
ADT data feeds containing the dated admissions, discharges, and transfer state changes of patients and demographics from a hospital information system

The amount of data received by the data ingestion module 110 may vary among embodiments. Also, information technology (IT) settings, interoperability barriers, and contractual agreements may be used as a basis for determining the source of information an organization can access, and consequently the level of accuracy of the extracted episodes and implementation of the data ingestion module 110.

In one embodiment, insurance claims may be queried by the data ingestion module 110. In another embodiment, EMRs and ADT databases may be queried by the data ingestion module 110 to augment the information available. In another embodiment, administrative databases may be queried in addition to the aforemented sources. The data ingestion module 100 may query the selected data sources to extract all the services received by the patients for whom the hospital or HCO is responsible and within the time-window as defined in the bundled payment agreement.

### Data Pre-Processing Module

The data pre-processing module 120 receives the data collected by the data ingestion module 110, either in response to a request from the data pre-processing module 120 or based on a pre-programmed transfer operation performed by the data ingestion module 110. The data pre-processing module 120 may then perform one or more dedicated pre-processing routines, for example, in order to cleanse the data feeds. The pre-processing routines are known and may be provided for each of a plurality of data types (e.g., claims, ADT, etc.).

Subsequently, the data pre-processing module 120 may merge the diverse data feeds and normalize them in order to generate one or more lists of unique services received by the selected patients within the selected time-window. Each entry in the list may indicate, for example, the date of service, the procedure rendered, all listed diagnoses, and/or reimbursed costs by the payer. Apache NiFi is an example of a technology that may be used to route and transform data from beginning to end. The lists and data generated by the data pre-processing module 120 may be input into the episode-grouper module 130, and/or may be input into a commercially available episode grouper.

### Episode-Grouper Module

The episode of care grouper (episode-grouper) module 130 may determine one or more episode types from the lists and data received from the data pre-processing module 120. The episode types may include ones for which a healthcare organization of a user has a bundled payment agreement. Additionally, or alternatively, a user may select which episode type should be extracted from the lists and data. The episode types may include chronic episodes and acute episodes. An example of a chronic episode of care is a patient diagnosed with heart failure and all services rendered to that patient since his/her diagnosis. An example of an acute episode of care is a joint replacement involving an arthroplasty procedure and all follow-up or rehabilitation services performed in the year after replacement surgery is performed.

In one embodiment, the episode-grouper module 130 generates a list of all episodes identified for all episode types selected or otherwise designated, and each episode is linked to a list of services corresponding to that episode. In one embodiment, the episode-grouper module 130 generates a list of all episodes identified for all the episode types selected, and each episode is linked to a list of services corresponding to that episode. In this case, each service in the list may be categorized as a typical service or a potentially avoidable service (PAS). One example of episode-grouper module 130 is disclosed at http://prometheusanalytics.net/deeper-dive/concepts-and-use.

### Episodic Care Pathway Analyzer Module

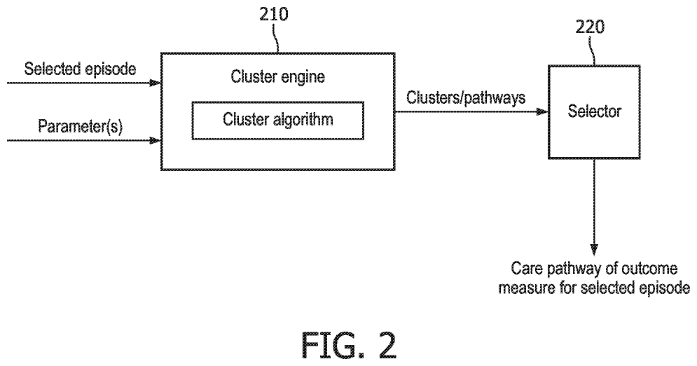

FIG. 2 illustrates an embodiment of the episode care pathway analyzer module 140, which includes a clustering engine 210 and a selector 220. The clustering engine 140 generates clusters for episode data using at least one predetermined algorithm 215 and based on one or more inputs. The inputs may include a selected episode and one or more parameters to be used by the algorithm 215 to generate a plurality of clusters. The selector 220 may select one of the clusters output from the clustering engine 210 as the cluster that satisfies (e.g., best achieves) the outcome measure for the selected episode. Because each cluster corresponds to a different care pathway, selection of a cluster by the selector 220 results in the selection of a care pathway for outcome measure of that episode.

The clustering engine 210 may use different algorithms to generate clusters and care pathways under different circumstances (e.g., based on different selected outcome measures, episodes, and/or other considerations) or may statically apply the same algorithm irrespective of outcome measure or episode selected.

In one embodiment, the clustering engine 210 may use a longitudinal clustering algorithm 215 to cluster episode data. Longitudinal clustering analysis is a type of unsupervised machine learning method which may be used to determine a set of homogeneous subgroups within an initial group of time-series. In one embodiment, the clustering engine may cluster episode data longitudinally, for example, in order to identify trends in cost. The clustering engine 210 may perform a clustering analysis for each of a plurality of episode types, which, for example, may be selected by an application, control software or a user.

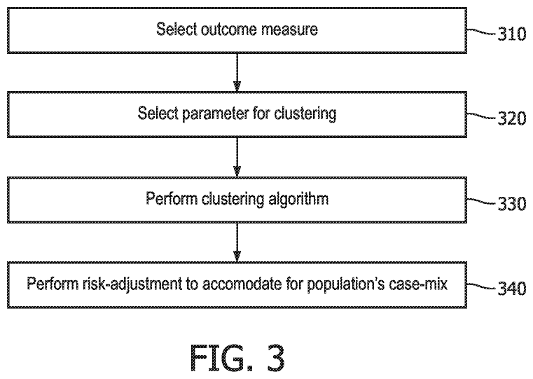

FIG. 3 shows an embodiment of operations performed by the clustiner engine 210 based on a longitudinal clustering algorithm. At 310, one or more outcome measures may be selected. At 320, and in this embodiment, the clustering engine may perform a longitudinal analysis based on selected parameters which include an outcome measure and a time-step. These parameters may be selected (e.g., by a user, application, or other control software) from the following operations set forth below. The selected options may then be input into the clustering engine to be used for generating a plurality of clusters for the selected episode and outcome measure.

| Outcome Measure | Time-Step |
|---|---|
| Total Cost, PAS Cost, or | Weekly, Monthly, Quarterly, |
| PAS Count | or Annually |

At 330, after the outcome measure and time-step are selected, one or more dedicated algorithms 215 for performing the longitudinal analysis compute clusters over the time per episode (indicated by the selected time step) for the lists generated for the episodes. In one embodiment, the clustering engine 210 may generate a plurality of clusters for each of a plurality of outcome measures for one or more selected episodes. The output of these algorithms may include information indicating a collection of time-series clusters, where each time-series corresponds to a care pathway within an episode and represents the evolution over time of the selected outcome. In this analysis, all outcome measures may be equally spaced over time, as defined by the time-step.

Examples of dedicated algorithm 215 that may be used to compute the clusters will now be discussed. In accordance with one example, the Episode Care Pathway Analyzer Module 140 groups the complete set of care pathways within an episode into subsets of care pathways with similar characteristics in cost trend or other pre-set attributes. This grouping can be performing using various techniques. For example, by using a distance measure that extends over time, care pathways can be grouped together. Any two care pathways that are closeby with respect to pair-wise distance are likely to be put into the same group, while any two care pathways that are further away are likely to be put in distinct groups.

To implement this technique, allow care pathways to be represented by an object oᵢ=(c₁, c₂, . . . , c_{T}) described by T values of cost (or any other relevant attribute) across various moments in time t=1 . . . T. These moments in time may be equidistant in real time. An example of a distance measure is the Euclidean distance, which measures by any two care pathways object oᵢ and oⱼ, though other distance measure can be used as well, as indicated by the following equation.

$$d ( {o}_{i} , {o}_{j} ) = \sqrt{{\sum }_{t = 1}^{T} {( {c}_{i , t} - {c}_{j , t} )}^{2}}$$

In one implementation, the cost values of the care pathways may be accessed or made available at equal (equidistant) times. If this is not the case, correction mechanisms may be implemented by incorporating a time difference correction in the distance measures, for example, using dynamic time warping or by applying linear piecewise approximation of the cost trends over time.

Clustering methods (e.g., agglomerative hierarchical clustering (AHC) or k-means clustering (KMC)) may be used to group a care pathway object based on the distance measure. AHC may identify the hierarchy of care pathway objects with respect to cost trend by starting bottom-up, for example, by comparing any two care pathway objects on their distance. KMC groups care pathway objects in a pre-determined number of groups and by minimizing the distance of objects within a group and maximizing the distances between groups.

Instead of distance-based grouping techniques, other methods include a method to estimate the parameters of a parametric model from the cost trend within all care pathways. These parameters may be used for clustering, e.g., by AHC or KMC. Parametric models include a curve model, a regression model, time series models (ARIMA), or mixed effects models. For instance, a curve model describes a set of cost trends of care pathway models in terms of a mean trend and random variations around this mean trend. The mean trend can be linear or first/second-order polynomial in time.

A linear trend of a care pathway i over time t=1 . . . T can be given by the following equation:

o_{i,t}=β_{0,i}+β_{1,i}Δₜ+ε_{i,t}

where o_{i,t} denotes the cost observation at time t, β_{1,i} and β_{1,i} denote the intercept and slope of the cost trend, respectively. The time component λₜ assesses the pre-specified linear change over time and the error ε_{i,t} is zero-mean, fixed variance, uncorrelated normally distributed. Various methods exist to estimate the slope and intercept (and possibly random effects on these parameters) that can be used as input values to the clustering method.

At 340, the outcome measures may be risk-adjusted to account for the case-mix of the population of patients under observation. This operation may be performed, for example, to eliminate, or attenuate as much as possible, any adverse impact on the outcome by factors (e.g. patient characteristics) not under control of the healthcare provider. When risk-adjustment is performed, any variability in the data may be attributable (with a relatively higher degree of accuracy) only to the quality of the care provided.

As previously indicated, the episode care pathway analyzer module 140 may analyze the time-series using a longitudinal clustering technique. In one implementation, a longitudinal k-means clustering analysis may be used. As an example, consider N care pathways (and hence N time-series), for a given episode, where each outcome measurement is denoted as xᵢⱼ, where i in {1, 2, . . . , N} and j in {1, 2, . . . , T} and where T is the number of measurements (time steps). The k-means algorithm may search for the clustering G=(g₁, g₂, . . . gₖ) that satisfies Equation (1):

$$arg {min}_{G} {\sum }_{i = 1}^{k} {\sum }_{x ∈ {g}_{i}} {‖ x - {\hat{μ}}_{i} ‖}^{2} \qquad ( 1 )$$

where \hat{μ}ᵢ denotes the centroids of cluster gᵢ. In this case, each episode x may be assigned to a cluster gᵢ, and each cluster gᵢ corresponds to a care pathway. The number of clusters K may initially be unknown, and therefore may be chosen beforehand using, for example, empirical methods. As shown in Equation (1), the care pathway corresponding to the cluster with the minimum value is selected as the optimal or best care pathway for the episode. This equation may be used when, for example, the outcome measure is the total cost over time.

A different equation may be used for a different outcome measure. For example, another outcome measure may be a summative score of patient experience throughout the care pathway expressing how satisfied a patient was during the various moments of time of care delivery. Care pathways may then be grouped for a particular diagnosis or treatment sessions on different trends of patient satisfaction levels. Other outcomes may relate to quality of care, e.g., patient waiting time after referral or between any two appointments, travelling distance of patients, number of clinical touch points between providers and patient, etc.

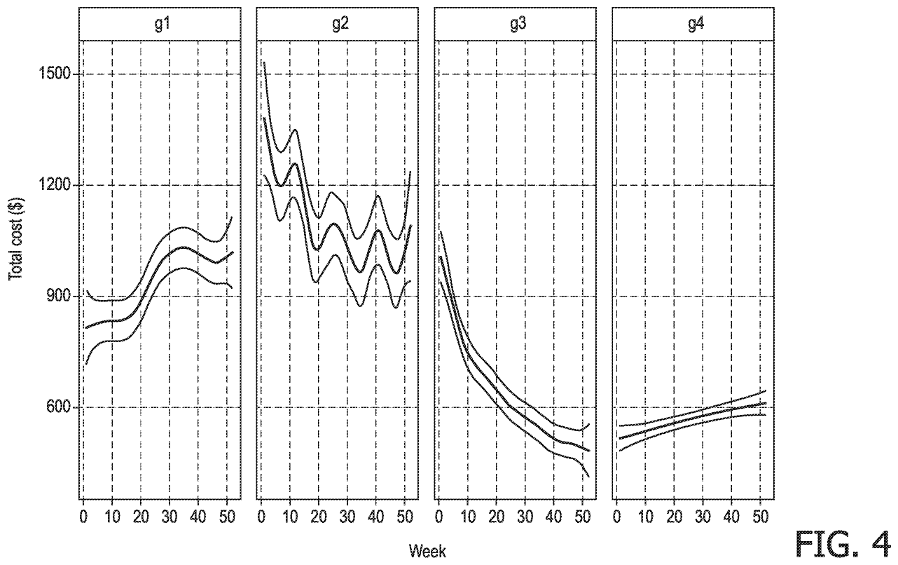

FIG. 4 illustrates an example of the results of a longitudinal cluster analysis performed by the the episode care pathway analyzer module 140 for data collected over a period of time. The results are expressed in panels corresponding to four clusters gᵢ to g₄, where K=4. Each cluster gᵢ corresponds to a different pathway. The panel for each cluster includes a smooth trajectory (or curve) representing the total cost for that cluster over time, which is 52 weeks in this example. The panel for each cluster also includes a shaded area that represents a predetermined statistical (e.g., 95%) confidence level relative to the smooth trajectory for the cluster.

As illustrated in the example of FIG. 4, the trajectories of the four clusters are very different. These differences provide indications of differences of the total cost per episode for each cluster. In the panel corresponding to cluster gᵢ, the trajectory of the curve shows a steady rise in total costs over time and that the costs are relatively substantial. In the panel corresponding to cluster g₂, the trajectory of the curve shows a very high initial total cost and then a wavering decrease in total costs over time. In the panel corresponding to cluster g₃, the trajectory of the curve shows a steep decline in total costs over time which results at the lowest cost level of the four clusters. In the panel corresponding to cluster g₄, the trajectory of the curve shows a slow rise in total costs over time, but the costs are at relatively low levels. The trajectories in the panels, therefore, demonstrate qualitative differences among total cost trends (e.g., outcome measure) evolving over time.

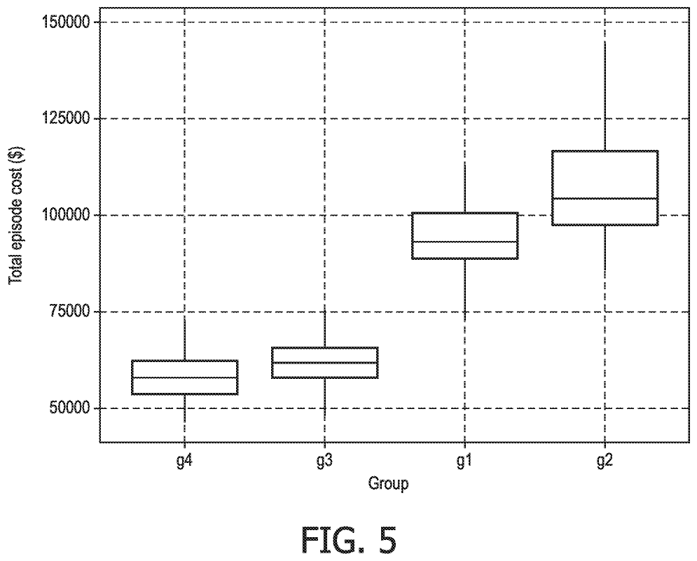

FIG. 5 illustrates a box plot of the mean (thick horizontal line) and distribution (box) of total episode cost for each cluster corresponding to FIG. 4. The clusters are arranged, from left to right, in the order of increasing mean total episode cost. Referring to FIG. 5, as the clusters gᵢ to g₄ are populated, the clustering engine 210 of the episode care pathway analyzer module 140 computes the total outcome measure for all the episodes for the entire period of study. For instance, when total cost is selected as the outcome measure, clustering engine 210 may compute the total episode cost the entire period of analysis. The mean total outcome measure for the episode may be computed for each cluster and may be used as a basis for ordering the clusters. In this example, four clusters are illustrated and ordered according to increasing mean total episode cost.

In other examples, the longitudinal clustering analysis may be performed for these and/or other outcome measures or trends (e.g., increasing costs, decreasing costs, overall low cost, a variable cost, etc.) may be compared for care pathways. Also, while patients may be treated for a similar condition within a given episode of care, a variety of services in follow-up care or rehabilitation may result in qualitatively different cost patterns, as illustrated by the trajectories in the four panels.

In one embodiment, the clustering engine 210 may cluster episode data based on a cross-sectional clustering algorithm 215 in order to identify, for example, mean level characteristics in cost. In implementing this algorithm, baseline and aggregate data of the episodes are collected and subjected to clustering. The data may report as variables on, for example, total cost of care, PAS cost, PAS count, quantity and quality of providers involved, count and type of services rendered, healthcare utilization (admission, ED visits), in/out-of-network services, care coordination level (e.g., ADT dates followed by appropriate follow-up community or primary care) for every pathway within an episode. Then, the following operations are performed. Examples of cross-sectional clustering algorithms that may be used are disclosed in Algorithms for Clustering Data, Anil K. Jain and Richard C. Dunes, Prentice Hall, 1988, and Data Clustering, Chandan K. Reddy, Charu C. Aggarwal, Chapman and Hall, 2016. An example of latent class analysis is disclosed in Applied Latent Class Analysis, (Eds.) Jacques Hagenaars and Alan McCutcheon, Cambridge University Press.

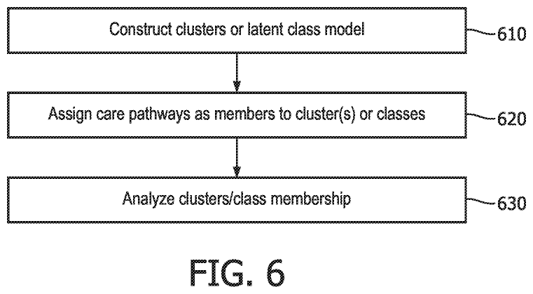

FIG. 6 illustrates operations which may be performed by the clustering engine 210 in order to implement the cross-sectional clustering algorithm for purpose of generating a plurality of clusters/care pathways and then selecting the care pathway that satisfies the selected outcome measure for a given episode.

In operation 610, one or more clusters and/or a latent class model is constructed for the variables. An example of the latent class model may be constructed as follows. Consider X to represent a latent variable and Y1 to represent any one of L observations. Then, a set a maximum number of latent classes, C, may be defined. For Y1, Y2 and Y3, dichotomous responses to observations may be determined for care coordination implemented yes or no, healthcare utilization high or low and PAS happened yes or no. The frequencies of occurrences of all response patterns for Y1, Y2 and Y3 are then enumerated. For three dichotomous response Y variables, there may be up to 8 different response patterns. The number of times every response patterns occurs may then be counted.

The latent class X allows different subgroups to be determined that deliver different quality of care from these response patterns. For example, consider starting from two levels of quality (C=2, being low or high). Various methods and tools may be used to find these latent classes (e.g., see https://www.statisticalinnovations.com/).

In operation 620, care pathways of episodes (e.g., patients or beneficiaries) are assigned as members to the clusters or latent classes based on posterior membership probabilities. This may be accomplished using a latent class model. For example, a latent class model may be used that has the basic notion that the probability of observing a response pattern y, P(Y=y), is a weighted average of the C class-specific probabilities P(Y=y|X=x); that is, P(Y=y)=\Sigma_({x=1}\hat{ }{C}P(X=x)P(Y=y|X=x). P(X=x) is considered the proportion of care pathways (patients) that belong to a particular latent class expressing quality of care delivered in our example. The conditional probability P(Y=y|X=x) can be computed as a product of the mutually independent conditional response probabilities \product_{l=1}\hat{ }{L}P(Y1=yl|X=x).

Using this model, patients (care pathways) may be assigned to latent classes using, for example, Bayes rule and estimating the posterior latent class membership probability. This may be used as a basis for identifying which patients (care pathways) are more likely to end up in a low or high quality of care cluster.

In operation 630, the assigned cluster/class membership is analyzed based on external variables (e.g., beneficiary characteristics) using cross-tabulation, stratification, regression, and/or other techniques. Using cross-tabulation, for example, the relationships between multiple variables may be determined based on categorical data. Cross-tabulation may be performed, for example, on a region, age group, or gender. In one embodiment, pivot tables may be used in spread sheets.

Using stratification, items (care pathways) may be divided into homogeneous sub-groups. These subgroups (strata) are mutually exclusive and all items may be covered by all subgroups exhaustively. In one example, the following may be stratified: risk or disease severity sub-groups, care pathways that cover low, medium or very sick patients.

Using regression, the relationship among independent variables to a single dependent variable or ‘criterion’ may be estimated. For example, the change in this dependent criterion variable may be determined when some of its dependent variables change. Such a regression may be performed, for example, based on age, income and ethnicity to a criterion named ‘social class.’ The social class criterion then be used to map out the various care pathways.

Longitudinal and cross-sectional clustering algorithms are examples of two approaches that may be used to generate and select clusters for determining a care pathway that satisfies an outcome for a given episode. In other embodiments, the clustering engine of module 140 may implement a different cluster analysis technique. For example, the clustering engine may implement one or more statistical methods, including but not limited to group-based trajectory modelling (GBTM), and growth mixture modelling (GMM). Examples of GBTM which may be used in accordance with one or more embodiments are disclosed in Nagin, D. S. (1999). Analyzing developmental trajectories: A semiparametric, group-based approach. Psychological Methods 4 139-157, Nagin, D. (2005). Group-based modeling of development. Harvard University Press, and GBTM is available through the TRAJ procedure in SAS, amongst other statistical packages. https://www.andrew.cmu.edu/user/bjones/. Examples of GMM are disclosed in GMM is a generalization of GBTM; It is available through several modeling programs, e.g. Mplus Muthen and Muthen (1998{2012), Latent GOLD (Vermunt and Magidson, 2016), and the R packages OpenMx (Boker et al., 2011) and 1 cmm (Proust-Lima, Philipps and Liquet, 2017).

### Insight Generator Module

The insight generator module 150 reports insights derived by the analysis performed by the episode care pathway analyzer module. This includes one or more of the following. Stratifying (longitudinally or cross-sectionally) clustered pathways in an episode of care may be performed for newly, previously assigned, or preliminary assigned beneficiaries on a periodic (e.g., quarterly or yearly) basis. For a healthcare organization (HCO) with CMS patients, it may be challenging to provide good quality care or to perform a total cost of care estimate to preliminary assigned beneficiaries until the beneficiaries are fully assigned at the end of the performance period (e.g., year). Newly assigned beneficiaries may be candidates to be enrolled in cost-effective pathways in an episode of care.

Stratifying (longitudinally or cross-sectional) clustered pathways in an episode of care may be performed based on a CMS-HCC risk score for beneficiaries to determine what pathways are followed by beneficiaries with different risk levels.

Stratifying (longitudinally or cross-sectionally) clustered pathways may be performed on providers that offer different levels of primary care services. Stratifying these pathways may be used as a basis for determining to what extent providers, who offer most primary care services, are represented in the most cost-effective pathways within an episode. If so, beneficiaries may be assigned to the primary care providers in favorable pathways.

Providing a (cross-)table per cluster, where all combinations of providers involved in the trajectories belonging to the cluster are listed and ordered based on the number of trajectories they were involved in. For instance, in the case of a simplified scenario (e.g., where only a surgeon and a physical physiotherapist are involved), the insight module 150 output may resemble Error! Reference source not found., where trajectories in a least cost-effective cluster are shown for different surgeon/physical physiotherapist pairs in the example under consideration.

TABLE 1

| | Physical | Number of |
|---|---|---|
| Surgeon | Physiotherapist | Trajectories |
| S1 | PP3 | 23 |
| S3 | PP2 | 12 |
| S4 | PP9 | 8 |
| . . . | . . . | . . . |
| S6 | PP1 | 1 |

For improvement purposes, some healthcare organizations may be interested in providers appearing at the top of the table for least cost-effective cluster. The insight module 150 may therefore perform a statistically based analysis that may be used by organizations to determine and deploy corrective initiatives to trigger improvements in the quality of care provided by the identified providers. In order to eliminate spurious appearances, a list of well-established provider pairs (or provider combinations, in case of more complex episodes) may be provided (e.g., by a user) when the module 15 is used to filter entries of the table. For instance, out-of-network providers may be excluded because, in some cases, the healthcare organization may not control the quality of the care, unless the organization is willing to engage with those providers through an affiliation.

Stratifying clustered pathways in an episode of care based on defined geographic areas around patient homes or other features of interest. In this case, a selected outcome may be attributed to those geographic strata (e.g., CPBSA, zip code area, counties, states). If episode costs are selected as the outcome of interest, paid amounts may be standardized to remove the effects of payment adjustments due to geographical concerns or policy considerations. In one embodiment, geographical cost weights may be used to adjust base rates for in-patient, out-patient, inpatient rehabilitation facility (IRF), long-term care hospital (LTCH), home healthcare agency (HHA), skilled nurse facility (SNF) and physician services payments. In one embodiment, a geographic benchmarking analysis may be performed to draw conclusions about care patterns and associated costs caused by differences in the local availability of providers. High cost trajectory clusters, for example, may be analyzed to point to geographic gaps in care, which may then be closed by targeted service delivery.

In one embodiment, the aforementioned stratification techniques may be replaced by cross-tabulation and/or regression methods.

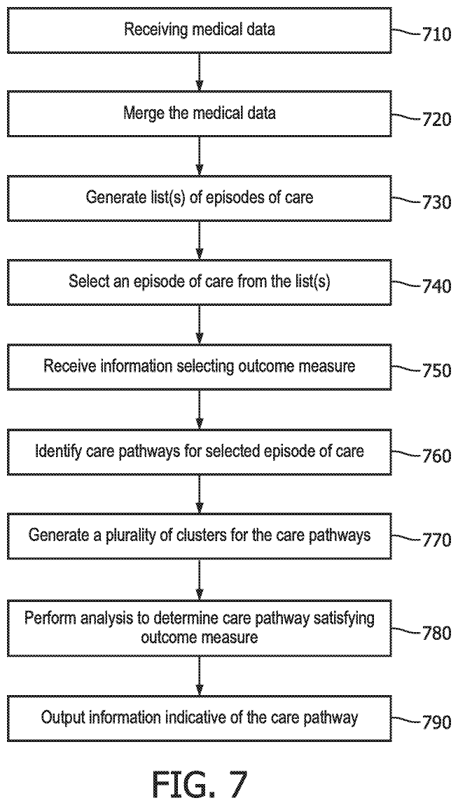

FIG. 7 illustrates operations included in one embodiment of a method for managing healthcare services. This method may be performed by the modules corresponding to the system embodiments (e.g., in FIG. 1) or may be performed by a different system.

In operation 710, the method includes receiving medical data. This operation may be performed, for example, by the data ingestion module 110 and the types of medical data received may include those types of information previously described in relation to data ingestion module 110.

In operation 720, the medical data may be merged or otherwise processed. This operation may be performed, for example, by the data pre-processing module 120 and may involve performing one or more dedicated pre-processing routines to cleanse the data feeds to the data ingestion module. Then, lists of services may be generated based on the medical data. This may involve, for example, an analysis of patient records, physical records, hospital and/or clinical records, insurance claims and/or other information.

In operation 730, one or more lists of episodes are generated, with each episode linked to the list of services corresponding to that episode. Each service in the list may be categorized, for example, as a typical service or a potentially avoidable service (PAS). Once the one or more lists of episodes are generated, an episode of care is selected from the list(s) of episodes generated in operation 720. These operations may be performed, for example, by the episode-grouper module 730. The episode types may include ones for which a healthcare organization of a user has a bundled payment agreement. In one embodiment, the episode types may include chronic episodes and acute episodes, examples of which have been previously discussed.

In operation 740, information is received that selects or otherwise designates one of a plurality of episodes of care. This information may be received from a user or may be generated by a program or computer according to an implementation of management software. In one embodiment, the present method may be successively performed for a plurality of selected episodes of care for comparison purposes and/or to otherwise arrive at an optimal pathway under a bundled payment reimbursement program. One of the modules or processor 50 in the system of FIG. 1 may receive and perform the operations of operation 730.

In operation 750, information is received (e.g., by processor 50 or one of the modules) selecting one of a plurality of outcome measures. The outcome measures may include, for example, total cost, PAS cost, or PAS count. In one embodiment, the outcome measure may be include qualify of patient care or healthcare services. The outcome measure may be selected by a user or may be selected based on predetermined information included in an application or other control software. At this time, information may also be received selecting a time step for the selected outcome measure. The time-step includes a period of time, e.g., weekly, monthly, quarterly, or annually.

In operation 760, a plurality of care pathways is identified for the selected episode of care. The operation may be performed, for example, episode care pathway analyzer module 140. Each of the care pathways may correspond to services derived from a combination of different healthcare personnel (e.g., primary care physician, specialists, clinical care personnel, etc.) and/or may correspond to a different combination of healthcare services, e.g., surgery, rehab, etc.

In operation 770, a plurality of clusters are generated to correspond to the plurality of care pathways, respectively. The clusters may generated by the episode care pathway analyzer module 140 as previously described, for example, based on time-series information as previously described for the longitudinal clustering algorithm.

In operation 780, a clustering analysis is performed to determine a care pathway corresponding to the cluster that satisfies the selected outcome measure for a bundled payment reimbursement contract. As previously described, this may involve performing a longitudinal clustering analysis or a cross-sectional clustering analysis, or alternatively may involve using a statistical model. This operation may also be performed by the episode care pathway analyzer module 140, as previously described.

In 790, information indicative of the care pathway that satisfies the selected outcome measure may be output, for example, to the display through an appropriate interface. This information may be output in a number of ways and, for example, may involve the use of a customized user interface for this purpose. In one embodiment, the care pathway that satisfies the selected outcome may be outputs as a graphical representation (e.g., trajectories, box plots, etc.), either alone or with other care pathways which have not be selected but which have bee output for comparison purposes. These operations may be performed by the insight generator module 150.

### Technological Innovation

One or more embodiments described herein address a problem and/or provide a technical solution to managing healthcare services and expenditures in a way not previously known or practiced. For example, one problem in the field is the inability to determine the best care pathway that satisfies a given outcome measure under a healthcare organization for a bundled payment reimbursement contract. No effective solution has been provided, especially achieving this purpose in the most cost-effective manner possible. The problem is exacerbated when it comes to providing hospital care.

A hospital or other healthcare organization faces the following challenges when choosing a bundled payment reimbursement scheme.
An inability to scale up in the number of patients, procedures, or conditions for which bundled payments are received. A solution to this problem would allow the hospital or organization to generate enough savings to justify the additional effort in care coordination.
An inability to select the best high-value providers to achieve good outcome without incurring in high costs. For instance, in surgery-like episodes, a recent study found that post-acute care costs might vary 4.8-fold and 4.3-fold for hip and knee replacement, respectively. Another study revealed that “In 2010, a quarter of nursing homes had an average Medicare length of stay of less than 24 days, while another quarter had a length of stay of more than 34 days”. Clinician expertise and patient volume for specialties or specific treatments are associated with better outcome.
An inability to control the variation in savings and healthcare spending to reduce waste and uncertainty in utilization in care delivery. For instance in Medicare Comprehensive Care for Joint Replacement (CJR) program 799 hospitals agreed on bundled payments for hip and knee surgery: 417 (52%) of the hospitals could not generate savings [3] and if a hospital could generate savings the analysis revealed a high variability in the incurred savings: mean episode savings varied from $13.83 to $3590.97.
An inability to understand patient freedom of choice and physician referral behaviour allowing to direct medical procedures and follow-up care to designated specialties and institutes.

Existing approaches have either failed to realize these problems or have been unable to solve them. As a result, HCO personnel are unable to make informed decisions as to the best way to allocate care versus cost, at least in a way that proves to be beneficial for both healthcare organizations and the patients they serve.

One or more embodiments described herein solve this problem by providing a system and method which determines the set of care pathways for a given episode of care that best satisfies an outcome measure selected by a hospital or HCO under a bundled payment reimbursement contract. This is achieved by providing a specific approach, using clustering algorithms and/or statistical models, in combination with other forms of analysis to determine the care pathway that satisfies an outcome measure of interest to an HCO. As a result, the embodiments therefore provide a solution which is not merely abstract in nature. Moreover, through the analysis performed by the disclosed embodiments, significantly more than merely the idea of managing healthcare services is performed, and at the very least the embodiments provide a practical application to healthcare resource allocation versus cost that provide real-world beneficial results.

In particular, one or more embodiments described herein allow:
1) HCOs to conduct a thorough analysis of clinical operations, to understand the feasibility and profit for any episode-based bundled payment scheme which they might be offered.
2) HCOs to partition all care pathways within an episode and determine the best possible pathway a patient should follow with respect to a specific episode, in order to achieve the best outcome, lowest cost and highest quality. This solves the problem of different patients within the same episode type receiving different types and volume of care, that leads to different quality, cost, and outcomes.
3) A benchmark to be provided for responsible physicians of patients that maybe used to identifying leading providers involved in the episodes diverging from the best trajectory. As a result, the best pattern (e.g., groups of pathways ‘behaving similarly’) may be identified within an episode.
4) HCOs to understand which programs to deploy for what episode of care in their own network. The embodiments herein may therefore cost-effectively manage specific episodes, while at the same time avoiding different outcomes and costs for patients within the same episode of care.
5) Care pathways to be partitioned and ranked for different outcome measures, in the specific area of bundled payment reimbursements. The outcome measures may involve cost, volume of care, qualify of care, variation in spending, and the type and nature of the outcome. Thus, the embodiments allow an HCO to navigate through the difficulty in making the best decisions for both the HCO and the patient.

Additionally, while one or more features of the embodiments may involve the use of a mathematical formula, the embodiments are in no way restricted solely to a mathematical formula. Nor are they directed to a method of organizing human activity or a mental process. Rather, the complex and specific approach taken by the embodiments, combined with the amount of information processing performed, negate the possibility of the embodiments being performed by human activity or a mental process. Moreover, while a computer or other form of processor may be used to implement one or more features of the embodiments, the embodiments are not solely directed to using a computer as a tool to otherwise perform a process that was previously performed manually.

Nor do these embodiments preempt the general concept of making healthcare cost decisions. For example, since the inception of bundled payment reimbursement contracts, healthcare organizations have allocated budgets to servicing patients. The embodiments described herein do not preempt, or otherwise restrict the public from practicing the general concept of, allocating healthcare resources. Rather, the embodiments take a specific approach (e.g., through clustering algorithms and the use of models) to achieve a specific purpose, e.g., a healthcare allocation solution customized to satisfy the differing interests of healthcare insurers, providers, and/or other health-related organizations. Moreover, one or more embodiments described herein may not focus on the definition of, nor the algorithm used to identify, an episode of care, and thus may be implemented in a manner agnostic to the algorithmic implementation deployed to define an episode of care. Moreover, the embodiments disclosed herein cannot be performed manually or by mental processes, for example, because the volume and dimensionality of the data being analyzed. The clustering techniques alone preclude manual or mental performance of the embodiments.

The methods, processes, and/or operations described herein may be performed by code or instructions to be executed by a computer, processor, controller, or other signal processing device. The code or instructions may be stored in a non-transitory computer-readable medium in accordance with one or more embodiments. Because the algorithms that form the basis of the methods (or operations of the computer, processor, controller, or other signal processing device) are described in detail, the code or instructions for implementing the operations of the method embodiments may transform the computer, processor, controller, or other signal processing device into a special-purpose processor for performing the methods herein.

The modules, models, engines, processors, and other information generating, processing, or calculating features of the embodiments disclosed herein may be implemented in logic which, for example, may include hardware, software, or both. When implemented at least partially in hardware, the modules, models, engines, processors, and other information generating, processing, or calculating features may be, for example, any one of a variety of integrated circuits including but not limited to an application-specific integrated circuit, a field-programmable gate array, a combination of logic gates, a system-on-chip, a microprocessor, or another type of processing or control circuit.

When implemented in at least partially in software, the modules, models, engines, processors, and other information generating, processing, or calculating features may include, for example, a memory or other storage device for storing code or instructions to be executed, for example, by a computer, processor, microprocessor, controller, or other signal processing device. Because the algorithms that form the basis of the methods (or operations of the computer, processor, microprocessor, controller, or other signal processing device) are described in detail, the code or instructions for implementing the operations of the method embodiments may transform the computer, processor, controller, or other signal processing device into a special-purpose processor for performing the methods herein.

It should be apparent from the foregoing description that various exemplary embodiments of the invention may be implemented in hardware. Furthermore, various exemplary embodiments may be implemented as instructions stored on a non-transitory machine-readable storage medium, such as a volatile or non-volatile memory, which may be read and executed by at least one processor to perform the operations described in detail herein. A non-transitory machine-readable storage medium may include any mechanism for storing information in a form readable by a machine, such as a personal or laptop computer, a server, or other computing device. Thus, a non-transitory machine-readable storage medium may include read-only memory (ROM), random-access memory (RAM), magnetic disk storage media, optical storage media, flash-memory devices, and similar storage media and excludes transitory signals.

It should be appreciated by those skilled in the art that any blocks and block diagrams herein represent conceptual views of illustrative circuitry embodying the principles of the invention. Implementation of particular blocks can vary while they can be implemented in the hardware or software domain without limiting the scope of the invention. Similarly, it will be appreciated that any flow charts, flow diagrams, state transition diagrams, pseudo code, and the like represent various processes which may be substantially represented in machine readable media and so executed by a computer or processor, whether or not such computer or processor is explicitly shown.

Accordingly, it is to be understood that the above description is intended to be illustrative and not restrictive. Many embodiments and applications other than the examples provided would be apparent upon reading the above description. The scope should be determined, not with reference to the above description or Abstract below, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

All terms used in the claims are intended to be given their broadest reasonable constructions and their ordinary meanings as understood by those knowledgeable in the technologies described herein unless an explicit indication to the contrary in made herein. In particular, use of the singular articles such as “a,” “the,” “said,” etc. should be read to recite one or more of the indicated elements unless a claim recites an explicit limitation to the contrary.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

We claim:

1. A method for managing healthcare services, comprising:
receiving information selecting an episode of care;
identifying a plurality of care pathways for the episode of care, each of the care pathways corresponding to services from a combination of different healthcare personnel with associated costs;
generating a plurality of clusters corresponding to the plurality of care pathways, respectively;
performing a clustering analysis to determine a first care pathway of the plurality of care pathways, the first care pathway corresponding to one of the plurality of clusters satisfying a selected outcome measure for a bundled payment reimbursement contract; and
outputting information indicative of the first care pathway.

2. The method of claim 1, wherein said performing include performing a clustering analysis to determine the first care pathway.

3. The method of claim 2, wherein performing the clustering analysis includes:
generating a plurality of trajectories for respective ones of the plurality of clusters,
wherein each trajectory corresponds to different combination of healthcare services or services from a different combination of healthcare personnel.

4. The method of claim 2, wherein the plurality of clusters is generated based on medical information derived from a plurality of healthcare resources.

5. The method of claim 1, wherein the clustering analysis includes performing a cross-tabulation, stratification, or regression algorithm on the plurality of clusters.

6. The method of claim 1, wherein the outcome measure includes at least one of volume, quality of care, cost, and variation in costs.

7. The method of claim 1, wherein:
the outcome measure includes cost of healthcare services over a period of time under the bundled payment reimbursement contract, and
the first care pathway corresponds to a lowest one of the total cost of the healthcare services.

8. The method of claim 1, wherein outputting information indicative of the first care pathway includes ranking the plurality of care pathways for the episode of care.

9. A system for managing healthcare services, comprising:
an interface; and
a processor configured to identify a plurality of care pathways for an episode of care, generate a plurality of clusters corresponding to the plurality of care pathways, respectively, perform a clustering analysis to determine a first care pathway of the plurality of care pathways, and output information through the interface indicative of the first care pathway, wherein each of the care pathways corresponds to services from a combination of different healthcare personnel with associated costs and wherein the first care pathway corresponds to one of the plurality of clusters satisfying a selected outcome measure for a bundled payment reimbursement contract.

10. The system of claim 9, wherein the processor is configured to perform a clustering analysis to determine the first care pathway.

11. The system of claim 10, wherein the clustering analysis includes:
generating a plurality of trajectories for respective ones of the plurality of clusters,
wherein each trajectory corresponds to different combination of healthcare services or services from a different combination of healthcare personnel.

12. The system of claim 9, wherein the clustering analysis includes a cross-tabulation, stratification, or regression algorithm on the plurality of clusters.

13. The system of claim 9, wherein:
the outcome measure includes cost of healthcare services over a period of time under the bundled payment reimbursement contract, and
the first care pathway corresponds to a lowest one of the total cost of the healthcare services.

14. A non-transitory machine-readable medium including instructions to cause a processor to:
identify a plurality of care pathways for an episode of care;
generate a plurality of clusters corresponding to the plurality of care pathways, respectively;
perform a clustering analysis to determine a first care pathway of the plurality of care pathways; and
output information indicative of the first care pathway
wherein each of the care pathways corresponds to services from a combination of different healthcare personnel with associated costs and wherein the first care pathway corresponds to one of the plurality of clusters satisfying a selected outcome measure for a bundled payment reimbursement contract.

15. The medium of claim 14, wherein the processor is configured to perform a clustering analysis to determine the first care pathway.

16. The medium of claim 15, wherein performing the clustering analysis includes:
generating a plurality of trajectories for respective ones of the plurality of clusters,
wherein each trajectory corresponds to different combination of healthcare services or services from a different combination of healthcare personnel.

17. The medium of claim 14, wherein:
the outcome measure includes cost of healthcare services over a period of time under the bundled payment reimbursement contract, and
the first care pathway corresponds to a lowest one of the total cost of the healthcare services.
